(19) United States
(12) Patent Application Publication
(10) Pub. No.: US 2005/0273041 A1
(43) Pub. Date: Dec. 8, 2005

(54) Tampon and tampon applicator

(75) Inventors: Thomas Ward Osborn III, Clifton, OH (US); Diana Lynne Gann, Lebanon, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/861,712

(22) Filed: Jun. 4, 2004

(51) Int. Cl. A61F 13/20

(52) U.S. Cl. 604/15

(57) ABSTRACT

A tampon applicator for positioning a tampon inside a vaginal cavity. The applicator includes an outer member disposed co-axially with an inner member. The outer member has an outer member length. The tampon has a tampon length. The inner member is slidable within the outer member. The inner member has an inner member length which is less than or equal to the tampon length.

### FIELD OF INVENTION

This invention relates to a tampon and tampon applicator providing low placement of a tampon within the vaginal cavity.

### BACKGROUND OF THE INVENTION

Tampon applicators have been used for inserting a tampon into a vaginal cavity to prevent leakage of menstrual fluids or other fluids. In the field of tampon applicators, “push” type tampon applicators are well known. These applicators generally comprise, in a simple form, a pair of coaxial hollow cylinders or tubes. The larger outer tube serves as a vaginal insertion tube and houses a tampon. The smaller inner tube serves as a plunger which ejects the tampon from the outer tube.

It has been recognized that tampon applicators of the type described above have certain deficiencies which can be readily apparent to many users of such devices. In general, the position of the tampon within the vaginal cavity can directly impact the tampon's efficiency of reducing and/or eliminating leakage. For example, conventional “push” type applicators can place a tampon too high in the vaginal cavity. The higher the tampon is placed, the greater the chance of leakage due to the tampon being above the major path of fluid flow. In other words, menstrual fluids can pass by the tampon without being absorbed.

In addition, if the position of the tampon is too high in the vaginal cavity, the tampon can extend into portions of the vaginal cavity, where, for reasons of the size and mechanical properties of the tampon, the tampon can be negatively affected by the vaginal organs, further reducing the tampon coverage of menses flow in the vaginal cavity. Moreover, current tampon applicators are designed to “push” the tampon out of the outer tube so that the tip of the tampon is substantially higher than the outer tube. This often causes the tampon to be deflected by the cervix, resulting in an off-centered position of the tampon, and, thus, in possible leakage from the vaginal cavity.

Alternatively, a tampon can be placed too low in the vaginal cavity. When the tampon is placed too low in the vagina cavity, the too low position of the tampon can cause bodily discomfort to the wearer because of the pressure exerted from the sphincter muscles against the tampon. Also, if the tampon is placed too low, accidental expulsion of the tampon is a highly undesired risk.

To solve the above problem of positioning the tampon, the relative dimensions of the tampon and applicator can be such to provide the desired depth of placement of the tampon in the vaginal cavity.

### SUMMARY OF THE INVENTION

The present invention provides a tampon applicator for positioning a tampon inside a vaginal cavity. The applicator includes an outer member disposed co-axially with an inner member. The outer member has an outer member length. The tampon has a tampon length. The inner member is slidable within the outer member. The inner member has an inner member length which is less than or equal to the tampon length.

### BRIEF DESCRIPTION OF THE DRAWINGS

FIG. 1 is a side view of an applicator of the present invention in the pre-expelled state.

FIG. 2 is a side view of the applicator of FIG. I in a partially expelled state where the inner member is fully engaged with the outer member.

FIG. 3 is a side view of the applicator of FIG. I depicting the relative dimensions of the applicator.

FIG. 4 is a side view of another embodiment of an applicator of the present invention.

FIG. 5 is a side view of another embodiment of an applicator of the present invention.

### DETAILED DESCRIPTION OF THE INVENTION

As used herein, the term “tampon” refers to any type of absorbent structure that can be inserted into the vaginal cavity or other body cavities for the absorption of fluid therefrom or for the delivery of active materials, such as medicaments or moisture. A tampon can be straight or non-linear in shape, such as curved along the longitudinal axis.

The tampon length is measured from the top of the tampon to the base of the tampon along a longitudinal axis. The base of the tampon does not include any overwrap, secondary absorbent member, or withdrawal cord which extends beyond the tampon's main absorbent material. A typical tampon is 5-20 mm wide corresponding to the largest cylindrical cross section. The width can vary along the length of the tampon.

As used herein, the terms “vaginal cavity,” “within the vagina,” and “vaginal canal” refer to the internal genitalia of the human female in the pudendal region of the body. The term “vaginal canal” is not intended to include the interlabial space including the floor of the vestibule.

The externally visible genitalia generally are not included within the term “vaginal canal” as used herein.

As used herein, the term “hymen ring” refers to the demarcation between the vaginal cavity and external genitalia and is identified by the position of the hymen or residual tissue of the hymen.

As used herein, the term “low placement” refers to a position of the tampon inside the vaginal cavity, wherein the tampon is positioned below the cervix.

The term “fully expelled” refers to the base of the tampon being disengaged from all portions of the tampon applicator when the inner member of the tampon applicator is fully engaged or fully plunged with the outer member of the tampon applicator. The base of the tampon does not include any overwrap, secondary absorbent member, or withdrawal cord which extends beyond the tampon's main absorbent material.

The term “partially expelled,” as used herein, means that the base of the tampon is partially retained within a portion of the applicator when the inner member of the tampon applicator is fully engaged or fully plunged with the outer member of the tampon applicator. The base of the tampon does not include any overwrap, secondary absorbent member, or withdrawal cord which extends beyond the tampon's main absorbent material.

The term “joined” or “attached” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element; i.e., one element is essentially part of the other element.

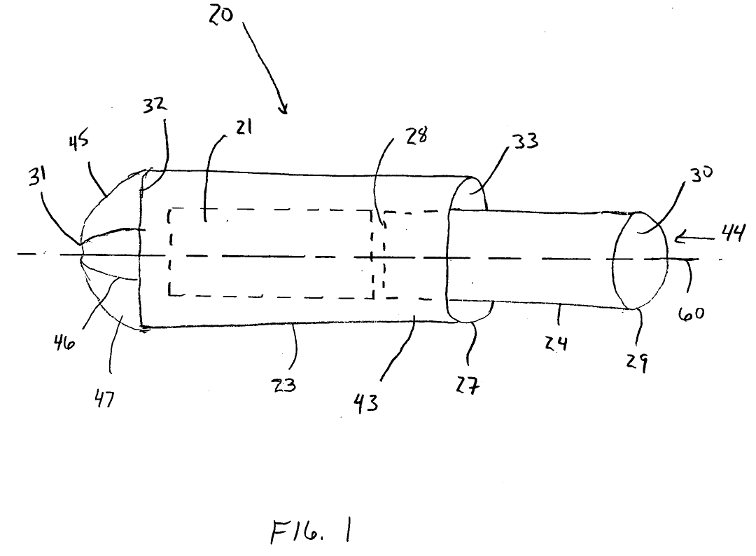

Referring to FIG. 1, a tampon applicator 20 is shown which is designed to position tampon 21 to achieve low placement inside the vaginal cavity. In addition, the present invention is directed to a tampon applicator 20 which partially expels tampon 21. The tampon applicator 20 is designed to house a tampon 21 and provide a comfortable means of inserting the tampon 21 into a woman's vagina.

Generally, the tampon applicator 20 includes an outer member 23 and an inner member 24. The outer member 23 comprises an insertion end 31 and a second end 27 opposed to the insertion end 31. The outer member 23 may comprise a preformed hinge or groove 32 extending around the periphery of the outer member 23 near the insertion end 31. The outer member 23 may also have a dome-shaped end 45 having a number of radial slits 46 therein extending from a central aperture to the groove 32. The outer member 23 may also have petals 47.

The inner member 24 has a first end 28 and a second end 29 opposed to the first end 28. The inner member 24 has a hollow interior portion 30. The inner member 24 is dimensioned to be slidabe within the hollow interior portion 33 of the outer member 23, with minimal clearance therebetween.

To use the tampon applicator 20 of the present invention the user will typically hold the grip region 43 near the second end 27 of the outer member 23, such as with her thumb and index finger, and insert the insertion end 31 of outer member 23 into the vaginal cavity. During insertion, the user applies a force 44 along longitudinal axis 60 strong enough to push the inner member 24 into the outer member 23. As a result, the inner member 24 slides toward the insertion end 31 of the outer member 23 pushing the tampon 21 toward the insertion end 31.

The tampon applicator 20 has a pre-expelled state and a partially expelled state. In the pre-expelled state, as is readily seen in FIG. 1, the tampon 21 sits within the outer member 23 and the inner member 24 is substantially aligned with the outer member 23. The tampon 21 can remain snugly therein without any outside force to sustain its position in the tampon applicator 20. The tampon 21 may or may not be in contact with the first end 28 of the inner member 24 before expulsion of the tampon 21.

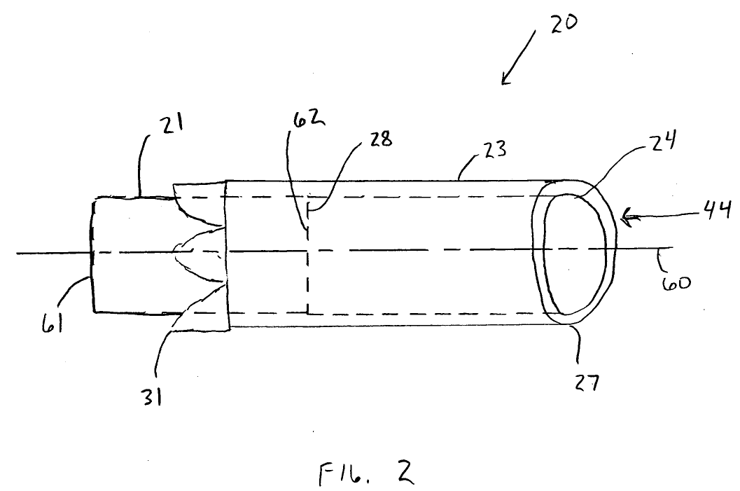

In the fully engaged state, as is readily seen in FIG. 2, the tampon 21 has been directionally expelled by the application of axial force 44 along the longitudinal axis 60. As force 44 is applied, the first end 28 of inner member 24 bears against the base 62 of tampon 21, pushing the first end 61 of tampon 21 toward the insertion end 31 of the outer member 23. When the axial force 44 is applied, the inner member 24 slides toward the insertion end 31 of the outer member 23 until the inner member 24 becomes fully engaged with the outer member 23. In the fully engaged state the tampon 21 is partially expelled out of the tampon applicator 20. During removal of the tampon applicator 20 from the body cavity, the body tissues hold onto the exposed portion of tampon 21 with the adherence of the absorbent surface to vaginal tissues and body pressure. Because the body tissues hold onto the exposed portion of tampon 21, the tampon applicator 20 exits the body cavity without the tampon 21, i.e., leaving the tampon 21 behind. This results in low placement of the tampon 21 within the vagina.

Tampon applicator 20 could be used to deliver any other type of solid object to any suitable cavity, in addition to delivering menstrual tampons to the vaginal canal.

Below will provide more detail of each component of the tampon applicator of the present invention.

i. Outer Member

Referring to FIG. 1, typically, the outer member 23 can be used to handle or grip the tampon applicator 20 during the insertion into the vaginal cavity. The outer member 23 is external to the inner member 24. The outer member 23 has a hollow interior 33, an insertion end 31, and a second end 27 opposed to the insertion end 31. The insertion end 31 is the portion of the outer member 23 in which the tampon applicator 20 is inserted into the vaginal cavity and it is the end from which the tampon 21 is expelled. The grip region 43 is the portion of the outer member 23 in which the user can handle or grip the tampon applicator 20. At least a portion of the hollow interior 33 of the outer member 23 can engage with at least a portion of the inner member 24. The inner member 24 is slidable within the outer member 23.

The manufacturer of the tampon applicator 20 can vary the size of the outer member 23. The size of the outer member 23 can be determined primarily by the dimensions of the tampon 21. Specifically, the diameter of the outer member 23 can be varied to accommodate different absorbency tampons. Generally, the outer member 23 can have an inner diameter of about 8 millimeters to about 21 millimeters and a wall thickness of about 0.4 millimeter to about 1.2 millimeter. Generally, the inner diameter of the outer member 23 should be suitably greater than the diameter of the tampon 21 to prevent the outer member 23 from interfering with the expulsion of the tampon 21 from the outer member 23. Moreover, the inner diameter of the outer member 23 can have varying diameters and shapes to conform to the profiled shape of the enclosed tampon 21.

Also, the manufacturer of the tampon applicator 20 can vary the length of the outer member 23. The length of the outer member 23 is measured from the insertion end 31 to the second end 27. Generally, the outer member 23 should be of a sufficient length to house the tampon 21 prior to the expulsion of the tampon 21 from the tampon applicator 20 into the vaginal cavity. Preferably, the outer member 23 completely houses the tampon 21 prior to expulsion.

In addition, the manufacturer of the tampon applicator 20 can vary the shape of the outer member 23. It is further noted herein that the shape of the outer member 23 can vary as long as a portion of the inner member 24 can be slideable within the outer member 23. Moreover, the outer member 23 is in no way limited by the shape that it can assume except that the shape should not hinder directional expulsion of the tampon 21. One skilled in the art can imagine that the outer member 23 might be cylindrical or curved like a banana or any other suitable shape as long as any such shape would work effectively to allow tampon 21 to expel from the tampon applicator 20, and comfortably insert into the vaginal cavity. The outer member 23 can be of any suitable cross-sectional shape. For example, suitable cross-sectional shapes can include, but are not limited to, circular, oval, flattened circular, elliptical, and any combination thereof.

Furthermore, the outer member 23 can contain the grip region 43 near the second end 27. The grip region 43 can provide for secure handling of the outer member 23. The perimeter of the grip region 43 can take essentially any desired shape, including oval, circular, and various other geometric forms. The gripping region 43 can be defined by outward projections or raised surfaces, created by impressing or compressing the surfaces. Referring again to FIG. 1, the grip region 43 is substantially circularly shaped, but it can also take on more angular formations such as squared. The surface of the grip region 43 can be any kind of surface known in the art. This surface can provide a desired frictional resistance for the fingers during the insertion of the tampon applicator 20 into the body. Moreover, this surface area can have surface texturing, impressions, and/or indentations.

Furthermore, it is preferable that the grip region 43 be of sufficient dimension to substantially provide a comfortable grip for the user. As used in the specification, the term “user's grip” means any way of holding the tampon applicator 20 in a hand, e.g., between a thumb and a finger. The grip region 43 need not necessarily extend completely around the perimeter of the outer member 23. For example, a space can be provided for a decorative marking or a character.

ii. Inner Member

The inner member 24 has a hollow interior 30, a first end 28, and a second end 29 opposed to the first end 28. The first end 28 is the portion of the inner member 24 which pushes against the tampon 21 during the expulsion of the tampon 21 from the outer member 23. The second end 29 is the portion of the inner member 24 in which the axial force 44 is applied to expel the tampon 21 from the outer member 23.

The manufacturer of the tampon applicator 20 can vary the shape of the inner member 24 as long as any such shape would work effectively to properly aide in the expulsion of the tampon 21 from the tampon applicator 20 and comfortably insert the tampon 21 into the vaginal cavity. One skilled in the art can imagine other shapes of the inner member 24, for example, cylindrical or curved like a banana or any other suitable shape possible. For example, the inner member 24 can be of any suitable cross-sectional shape including, but are not limited to, circular, oval, flattened circular, elliptical, and any combinations thereof.

The diameter of the inner member 24 can be varied to accommodate different absorbency tampons. For example, higher absorbency tampons can have larger diameters resulting in the inner diameter of the inner member 24 having a larger diameter. In addition, the inner diameter of the inner member 24 can have varying diameters and shapes to conform to the profiled shape of the outer member 23.

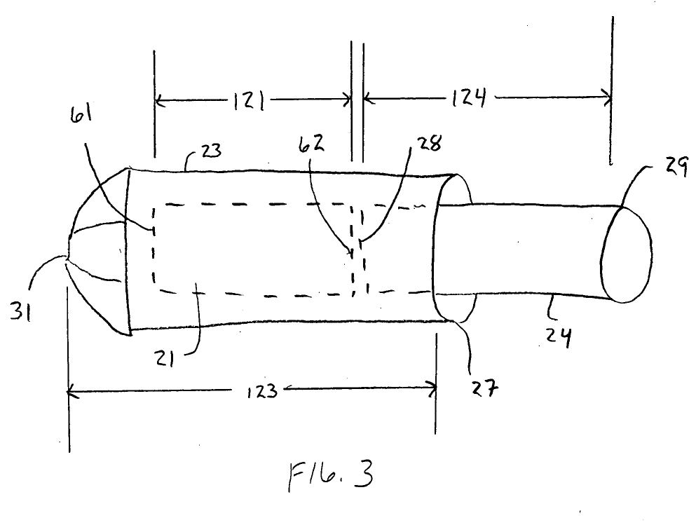

Referring now to FIG. 3, the length of the inner member 24 relative to the length of the tampon 21 is critical to provide proper low placement of the tampon 21 in the vaginal cavity. The inner member 24 must have an inner member length 124 which is less than or equal to the tampon length 121. The inner member length 124 is measured from the first end 28 to the second end 29. The tampon length 121 is measured from the first end 61 to the base 62. The outer member length 123 is measured from the insertion end 31 to the second end 27. For example, if the tampon length 121 is 50 millimeters, the inner member length 124 must be less than or equal to 50 millimeters. In such a configuration, if the tampon length is 50 millimeters an inner member length of 48 millimeters is acceptable.

An example of an applicator and tampon of the present invention is one where the outer member length is 70 mm, the inner member length is 58 mm and the tampon length is 46 mm. Another example of an applicator and tampon of the present invention is one where the outer member length is 66 mm, the inner member length is 52 mm and the tampon length is 42 mm. Another example of an applicator and tampon of the present invention is one where the outer member length is 60 mm, the inner member length is 44 mm and the tampon length is 44 mm.

By keeping the inner member length 124 less than or equal to the tampon length 121 any tampon 21 that is completely housed within an outer member 23 will be partially expelled from the outer member 23 upon complete or full engagement of the inner member 24 with the outer member 23. The outer member length 123 is greater than the tampon length 121. Thus, the tampon 21 is completely housed within the outer member 23. Upon complete engagement of the inner member 24 with the outer member 23 the tampon 21 will be partially expelled from the outer member 23 as is shown in FIG. 2.

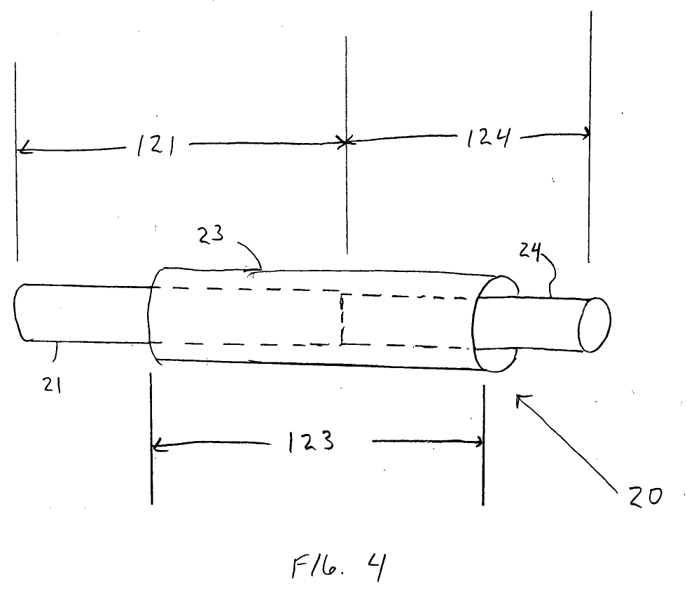

Referring now to FIG. 4, there is shown another embodiment of the tampon 21 and applicator 20 of the present invention. As can be seen, the tampon 21 is only partially housed within the outer member 23 leaving a portion of the 21 exposed. By keeping the inner member length 124 less than or equal to the tampon length 121 and less than the outer member length 123, tampon 21 will be partially expelled from the outer member 23 upon complete engagement of the inner member 24 with the outer member 23.

The less length of the tampon 21 that is exposed after expulsion from the tampon applicator 20, the lower the tampon 21 is placed in the vaginal cavity when compared to an applicator of equal length when the tampon is fully expelled. When the tampon 21 is placed low in the vaginal cavity there is greater coverage of vaginal cavity because the vaginal cavity is typically narrower in the lower region.

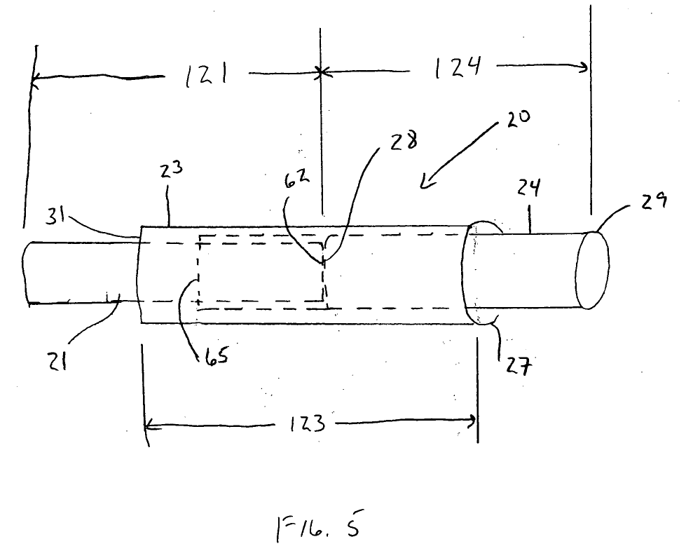

Referring now to FIG. 5, there is shown a tampon applicator 20 comprising an outer member 23 and an inner member 24. A tampon 21 is housed within applicator 20. Inner member 24 has a first end 28 and a second end 29. A portion of inner member 24 extends beyond first end 28 toward the insertion end 31 of outer member 23. The first end 28 of inner member 24 is determined by the position of tampon base 62 when tampon 21 is engaged by inner member 24. The inner member length 124 is less than or equal to the tampon length 121. The inner member length 124 is less than the outer member length 123. Upon full engagement of the inner member 24 with the outer member 23, the tampon 21 will be held by inner member 24 and a portion of tampon 21 will extend beyond the end 65 of inner member 24. Thus, upon fully engagement of inner member 24 with outer member 23, tampon 21 will be partially expelled from applicator 20.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A tampon applicator for positioning a tampon inside a vaginal cavity, said applicator comprising:
i. an outer member, said outer member having an outer member length;
ii. said tampon having a tampon length; and
iii. an inner member being slidable within said outer member, said inner member having an inner member length which is less than or equal to said tampon length.

2. The tampon applicator according to claim 1 wherein said outer member length is greater than said tampon length.
